(19) United States
(12) Patent Application Publication
(10) Pub. No.: US 2009/0054987 A1
(43) Pub. Date: Feb. 26, 2009

(54) SPINAL FUSION ASSEMBLY

(75) Inventors: Kingsley R. Chin, Philadelphia, PA (US); Christopher A. Chang, Beverly, MA (US)

Correspondence Address: AKC PATENTS, 215 GROVE ST., NEWTON, MA, 02466, US

(73) Assignee: SPINEFRONTIER LLS, Philadelphia, PA (US)

(21) Appl. No.: 11/779,470

(22) Filed: Jul. 18, 2007

Related U.S. Application Data
(63) Continuation of application No. 60/832,468, filed on Jul. 21, 2006.
Application No. 60/832,468 is a continuation-in-part of application No. 11/174,712, filed on Jul. 5, 2005.

(51) Int. Cl. A61F 2/44

(52) U.S. Cl. 623/17.16; 623/17.11

(57) ABSTRACT

An improved interbody spinal fusion assembly for fusing a first vertebra to a second vertebra includes an interbody cage, a first plate and a second plate. The interbody cage is configured to be implanted between the first and second vertebras and comprises through-apertures configured to be filled with bone fusing material. The first plate comprises an L-shaped body having a first end configured to be attached to a first location of the interbody cage, a second end configured to be attached to a first location of the first vertebra and a corner formed between the first and second ends and configured to be attached to a second location of the first vertebra or a third location of the interbody cage. The second plate comprises a first end configured to be attached to a second location of the interbody cage and a second end configured to be attached to a first location of the second vertebra.

### CROSS REFERENCE TO RELATED CO-PENDING APPLICATIONS

This application claims the benefit of U.S. provisional application Ser. No. 60/832,468 filed Jul. 21, 2006 and entitled “SYSTEM AND METHOD FOR FACET FIXATION”, the contents of which are expressly incorporated herein by reference.

This application is also a continuation in part of U.S. application Ser. No. 11/174,712 filed on Jul. 5, 2005 and entitled “METHOD AND DEVICE FOR KINEMATIC RETAINING CERVICAL PLATING” the contents of which are expressly incorporated herein by reference.

### FIELD OF THE INVENTION

The present invention relates to a spinal fusion assembly, and more particularly to a spinal fusion assembly including an interbody cage and a plate system configured to secure the interbody cage to adjacent vertebras.

### BACKGROUND OF THE INVENTION

The human spine consists of individual vertebras (segments) that are connected to each other. Under normal circumstances the structures that make up the spine function to protect the neural structures and to allow us to stand erect, bear axial loads, and be flexible for bending and rotation. However, disorders of the spine occur when one or more of these spine structures are abnormal. In these pathologic circumstances, surgery may be tried to restore the spine to normal, achieve stability, protect the neural structures, or to relief the patient of discomfort. The goal of spine surgery for a multitude of spinal disorders especially those causing compression of the neural structures is often decompression of the neural elements and/or fusion of adjacent vertebral segments. Fusion works well because it stops pain due to movement at the facet joints or intervertebral discs, holds the spine in place after correcting deformity, and prevents instability and or deformity of the spine after spine procedures such as discectomies, laminectomies or corpectomies. Discectomy and fusion or corpectomy and fusion are most commonly performed in the cervical spine but there is increasing application in the thoracic and lumbar spine, as well.

One way of achieving fusion of adjacent vertebras after discectomy is to insert an interbody cage implant carrying fusion promoting material between the adjacent vertebras. However, in some cases the interbody cage may slip out of place or the fusion promoting material may be dislodged causing interference with neighboring neurovascular structures and complications requiring follow-up surgeries. Accordingly there is a need for an improved interbody cage that addresses the above-mentioned limitations.

### SUMMARY OF THE INVENTION

In general, in one aspect, the invention features an interbody spinal fusion assembly for fusing a first vertebra to a second vertebra, where the first vertebra is adjacent to the second vertebra. The assembly includes an interbody cage, a first plate and a second plate. The interbody cage is configured to be implanted between the first and second vertebras and comprises through-apertures configured to be filled with bone fusing material. The first plate comprises an L-shaped body having a first end configured to be attached to a first location of the interbody cage, a second end configured to be attached to a first location of the first vertebra and a corner formed between the first and second ends and configured to be attached to a second location of the first vertebra or a third location of the interbody cage. The second plate comprises a first end configured to be attached to a second location of the interbody cage and a second end configured to be attached to a first location of the second vertebra.

Implementations of this aspect of the invention may include one or more of the following features. The first location of the interbody cage coincides with the second location of the interbody cage. The second plate comprises an I-shaped body or a T-shaped body. The second plate comprises a third end configured to be attached to a second location of the second vertebra. The second plate comprises an H-shaped body, V-shaped body, L-shaped body or X-shaped body. The spinal fusion assembly may further include a third plate comprising a first end configured to be attached to a third location of the interbody cage and a second end configured to be attached to a third location of the second vertebra. The interbody cage comprises a hollow body having a front insertion wall, a back trailing wall, left and right side walls and an X-shaped structure placed in the hollow body and having ends connected to locations in the side walls and the front and back walls. The interbody cage comprises an aperture configured to engage a tool used to insert the interbody cage between the first and second vertebras and the aperture is formed in one of the walls or at a corner between two of the walls. The through-apertures of the interbody cage are formed between the X-shaped structure and the walls. The interbody cage comprises top and bottom surfaces having protrusions configured to engage surfaces of the first and second vertebras. The protrusions may be ridges, grooves, teeth, serrations, or spikes. The interbody cage is made of a material including metal, ceramic, bone, PEEK, plastic, stainless steel, titanium, gold, silver, nickel, alloys thereof, polymer, composites, absorbable material, metal matrix material, polycarbon coating or combinations thereof. The bone fusing material may be hydroxyapatite, hydroxyapatite tricalcium, fibronectin, morphogenic proteins, bone growth promoting material, or combinations thereof. The spinal fusion assembly may further include one or more screws for attaching the plates to the interbody cage and the vertebras. The interbody cage is inserted anteriorly or posteriorly between the vertebras. The interbody cage comprises a bullet shaped front insertion wall. The interbody cage may have a width smaller than its height. The interbody cage may haves a width larger than its height. Any of the plates may have an adjustable length.

In general in another aspect the invention features a method for fusing a first vertebra to a second vertebra. The method includes first inserting an interbody cage between the first and second vertebras. Next, providing a first plate comprising an L-shaped body having first and second ends and a corner formed between the first and second ends and attaching the first end to a first location of the interbody cage, the second end to a first location of the first vertebra and the corner to a second location of the first vertebra or a third location of the interbody cage. Next, providing a second plate comprising first and second ends and attaching the first end to a second location of the interbody cage and the second end to a first location of the second vertebra. The first vertebra is adjacent to the second vertebra and the interbody cage comprises through-apertures configured to be filled with bone fusing material.

In general in another aspect the invention features an interbody spinal fusion assembly for fusing a first vertebra to a second vertebra, where the first vertebra is adjacent to the second vertebra. The assembly includes an interbody cage, a first plate and a second plate. The interbody cage is configured to be implanted between the first and second vertebras, and includes through-apertures configured to be filled with bone fusing material. The first plate comprises a body having a first end configured to be attached to a first location of the interbody cage and a second end configured to be attached to a first location of the first vertebra. The second plate comprises a first end configured to be attached to a second location of the interbody cage and a second end configured to be attached to a first location of the second vertebra. The first location of the interbody cage coincides with the second location of the interbody cage.

Among the advantages of this invention may be one or more of the following. The first and second plates are modular units that have various shapes and sizes and can be arranged to form various attachment configurations. This modular property of the plates allows them to be placed so that they don't interfere with the neighboring neurovascular structures and prevents potential injury of the neurovascular structures. The small size of the plates allows them to be implanted and removed one piece at a time via minimally invasive surgery.

### BRIEF DESCRIPTION OF THE DRAWINGS

Referring to the figures, wherein like numerals represent like parts throughout the several views:

FIG. 1 is a front perspective view of a an anterior lumbar interbody fusion (ALIF) assembly;

FIG. 2 is an exploded view of the ALIF assembly of FIG. 1;

FIG. 3 is a front perspective view of the interbody cage of FIG. 1;

FIG. 4A depicts a top view of the interbody cage of FIG. 3;

FIG. 4B depicts a front view of the interbody cage of FIG. 3;

FIG. 4C depicts a side view of the interbody cage of FIG. 3;

FIG. 5 is a front perspective view of an alternate embodiment of the interbody cage of FIG. 1;

FIG. 6A depicts a top view of the interbody cage of FIG. 5;

FIG. 6B depicts a front view of the interbody cage of FIG. 5;

FIG. 6C depicts a side view of the interbody cage of FIG. 5;

FIG. 7 is a front perspective view of another embodiment of the interbody cage of FIG. 1;

FIG. 8A is a side perspective view of a posterior lumbar interbody fusion (PLIF) assembly;

FIG. 8B is a front view of the PLIF assembly of FIG. 8A;

FIG. 9A is a back perspective view of the interbody cage of FIG. 8A;

FIG. 9B is a front perspective view of the interbody cage of FIG. 8A;

FIG. 10A depicts a top view of the interbody cage of FIG. 9A;

FIG. 10B depicts a back view of the interbody cage of FIG. 9A;

FIG. 10C depicts a side view of the interbody cage of FIG. 9A; and

FIG. 11A-FIG. 11B, FIG. 12A-FIG. 12B and FIG. 13 depict additional embodiments of the interbody fusion assembly.

### DETAILED DESCRIPTION OF THE INVENTION

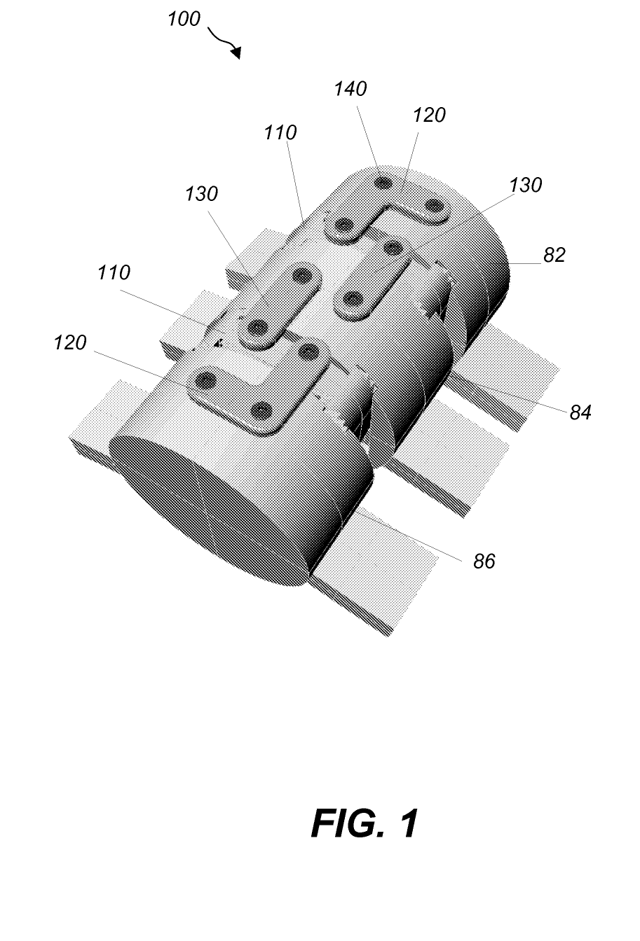

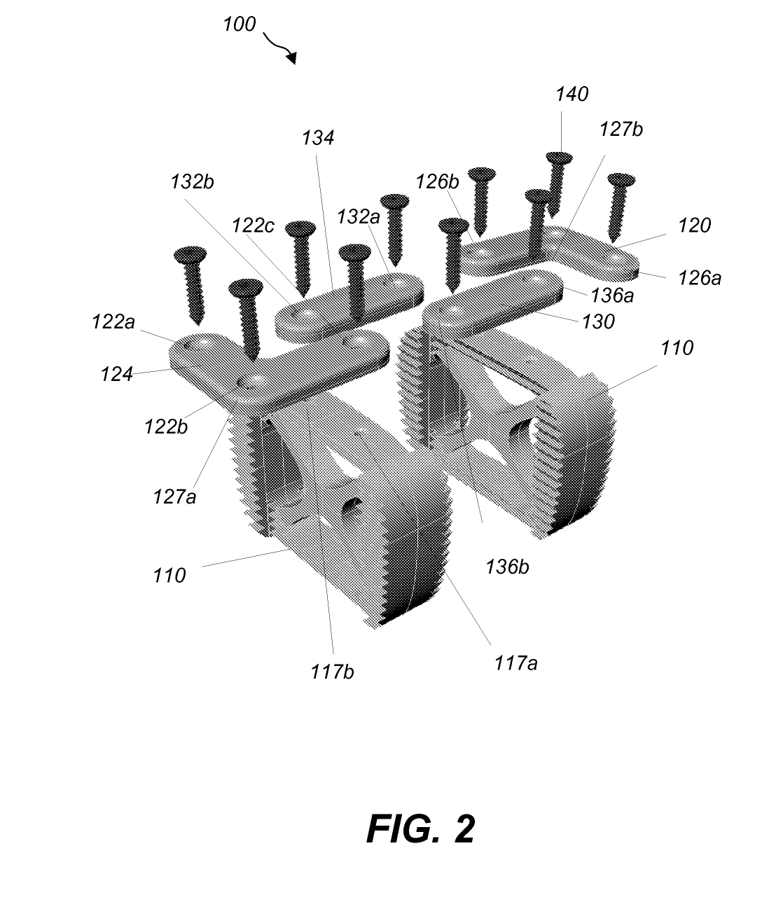

Referring to FIG. 1, an anterior lumbar interbody fusion (ALIF) assembly 100 includes an intervertebral implant or interbody cage 110 inserted in the disc space between two adjacent vertebras 82 and 84 and two plates 120, 130 securing the interbody cage 110 to the vertebras 82, 84, respectively, with screws 140. Referring to FIG. 2, plate 120 has an L-shaped flat body 124 having ends 126a, 126b. All ends 126a, 126b and corners 127a, 127b are rounded and the body 124 has three through-apertures 122a, 122b, 122c, configured to receive screws 140 for attaching end 126a and corner 127a of the plate 120 to the vertebra 82 and end 126b to the interbody cage 110. Plate 130 has an elongated flat body 134 having rounded ends 136a, 136b. Ends 136a, 136b include through-apertures 132a, 132b, configured to receive screws 140 for attaching ends 136a, 136b to the interbody cage 110 and the adjacent vertebra 84, respectively. In other embodiments plates 120, 130 are T-shaped, H-shaped, I-shaped, X-shaped or V-shaped (not shown). The purpose of these modular plates is to attach the interbody cage to the adjacent vertebras without interfering with adjacent neurovascular structures and without blocking the line of sight during surgery. The modular property of the plates allows them to be positioned in almost any possible orientation and to form any possible attachment configuration by combing two or more similar or different shaped plates together. In one example, plates 120, 130 have a length of 20 millimeters, a width of 9 millimeters and a thickness of 1 millimeter. The small size of the plates allows them to be implanted and removed one piece at a time via minimally invasive surgery. Plates 120, 130 may be made of metal such as stainless steel or titanium, plastic, bioabsorbable material and ceramic.

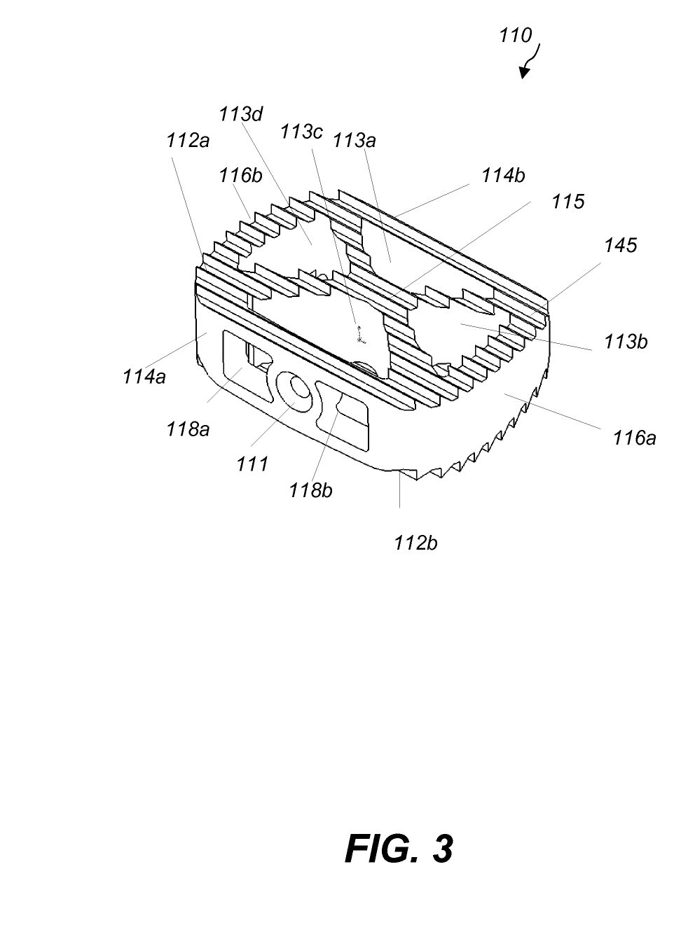

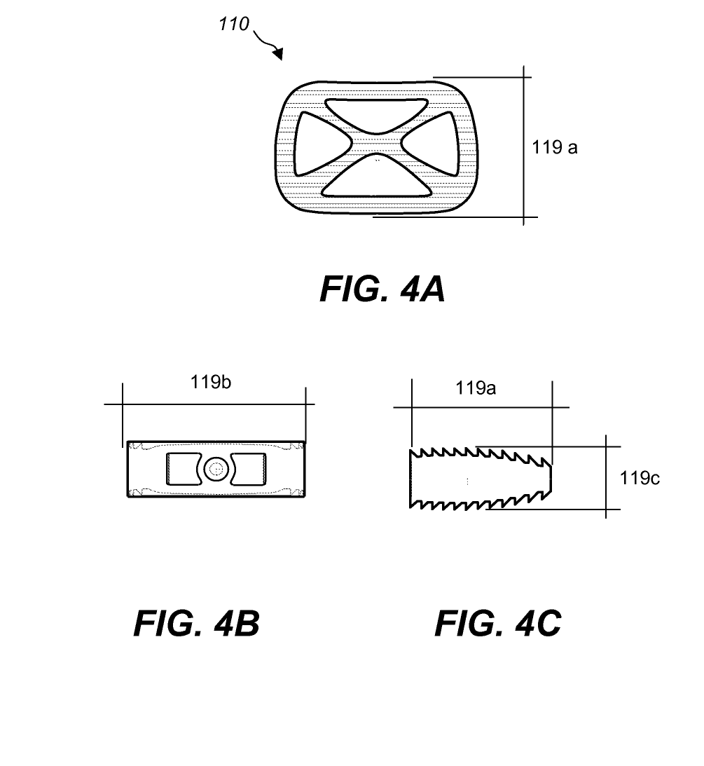

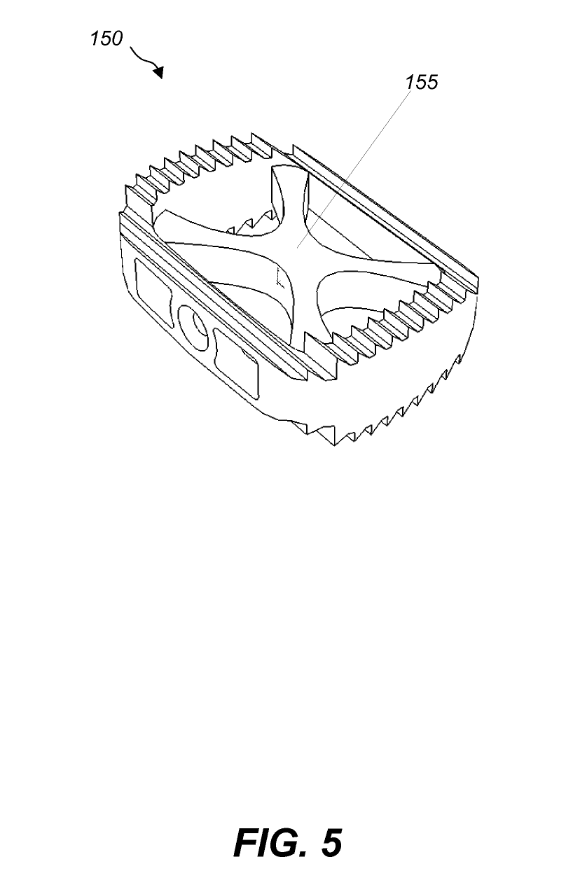

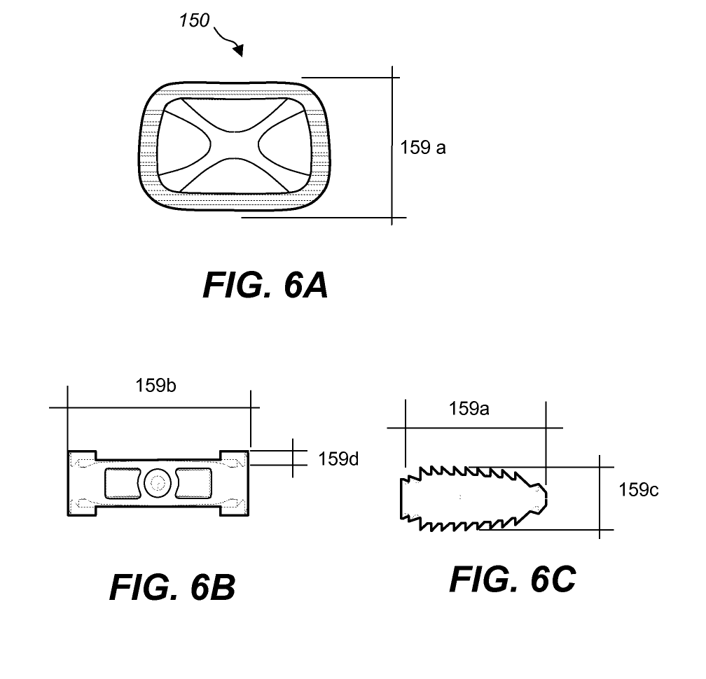

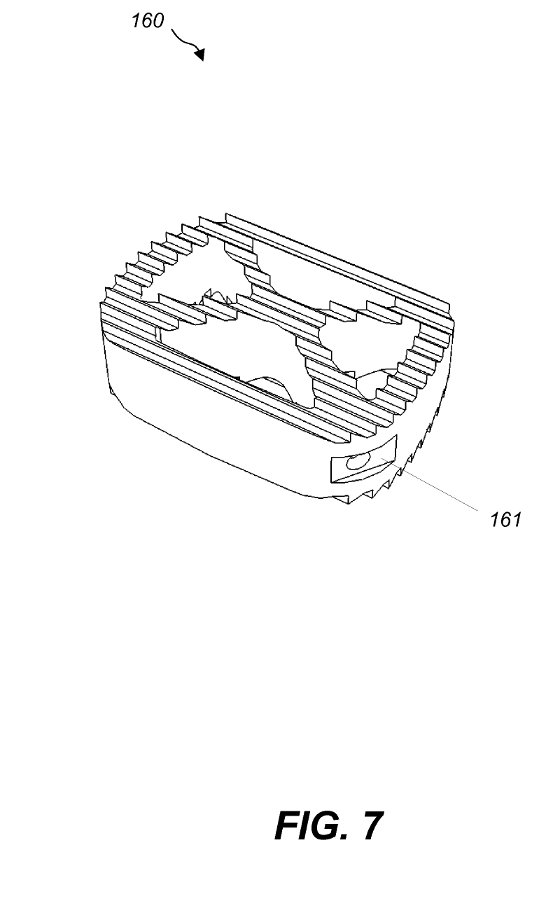

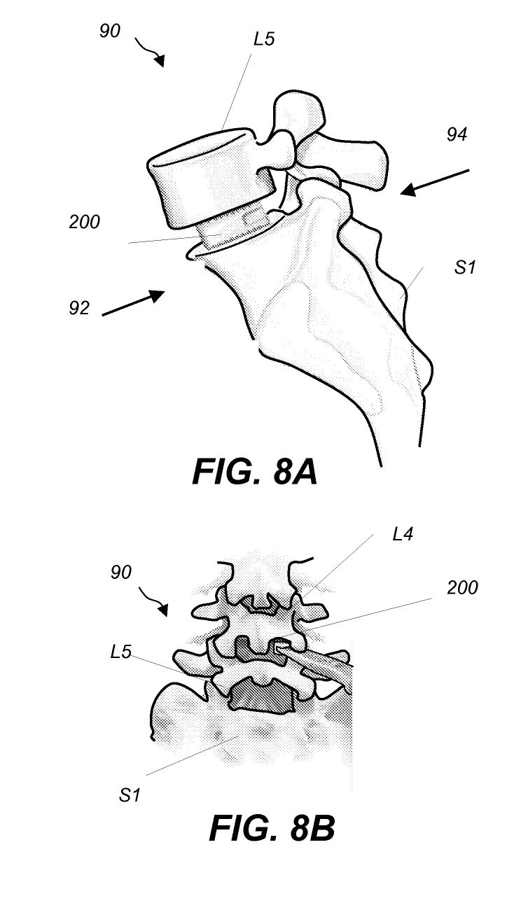

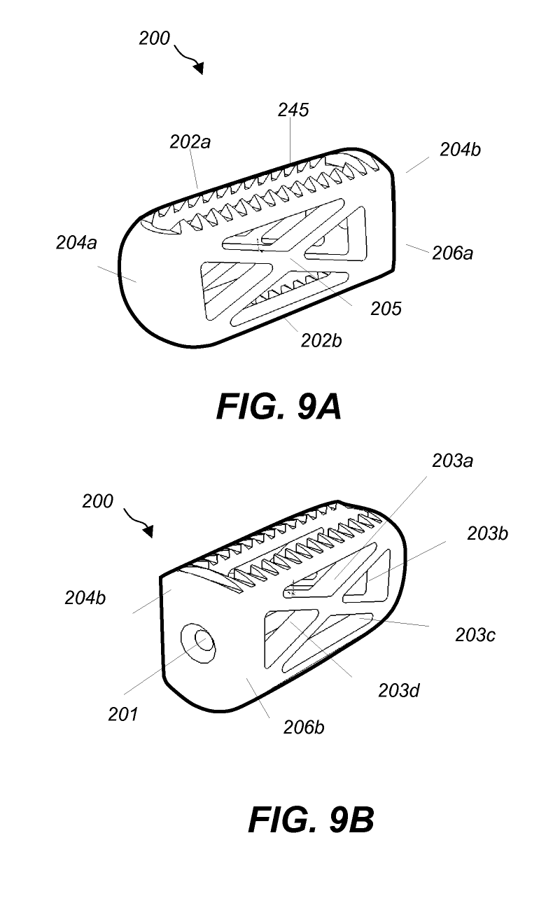

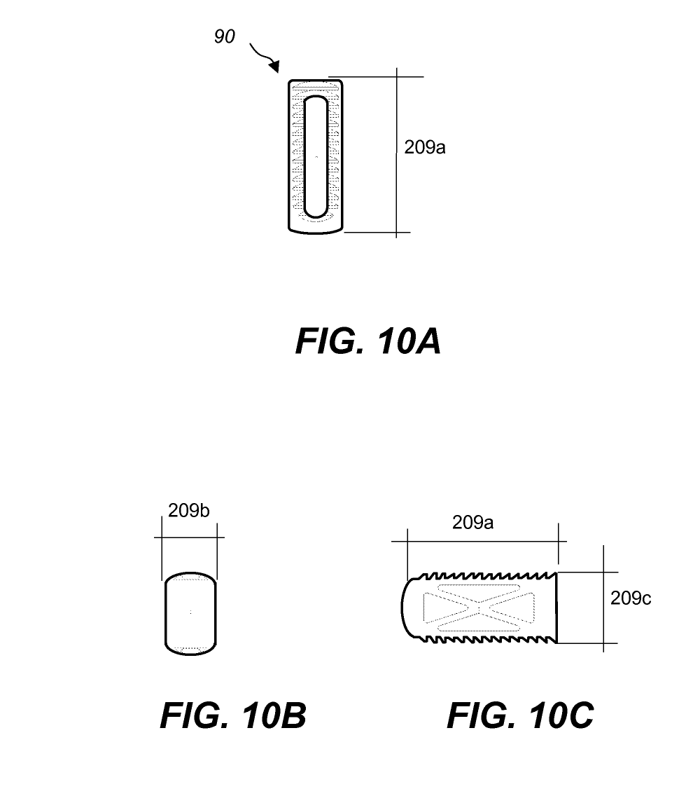

Referring to FIG. 2, FIG. 3, FIG. 4A-FIG. 4C, interbody cage 110 has a generally rectangular disk shape having a top surface 112a, a bottom surface 112b a front surface 114a, a back surface 114b, a right side surface 116a and a left side surface 116b. The inner body of cage 110 is basically hollow and includes an X-shaped structure 115 connecting the left side to the right side and the front side to the back side, as shown in FIG. 3. The X-shaped structure adds strength and flexibility to the overall interbody cage structure. Through apertures 113a, 113b, 113c, 113d are formed between the X-shaped structure 115 and the side, front and back structures, as shown in FIG. 3. The top and bottom surfaces have surface teeth 145 used to engage the adjacent vertebral surfaces. In other embodiments surfaces 112a, 112b include ridges, serrations, grooves, or spikes. The front surface includes fenestrations 118a, 118b extending through to the center of the structure and a threaded aperture 111 used for engaging a tool for positioning the interbody cage 110 between the vertebras 82, 84. Within apertures 113a-113d, material that promotes bone growth is deposited. Bone growth promoting material includes autogenous bone material, artificial osteogenic or osteoconducting material, or other fusion enhancing material. Examples of these material include bone harvested from the patient, hydroxyapatite, hydroxyapatite tricalcium or bone morphogenic proteins, among others. The interbody cage may be made of metal such as metal, ceramic, bone, PEEK, plastic, stainless steel, titanium, gold, silver, nickel, alloys thereof, polymer, composites, absorbable material, metal matrix material, polycarbon coating or combinations thereof. In one example the interbody cage 110 is made of Polyetheretherketone (PEEK) and has a length 119a about 19 millimeters, width 119b about 26 millimeters and height 119c about 9 millimeters. The side cross-section has a wedge shape converging toward the back side, as shown in FIG. 4C. In some embodiments, teeth 145 cover only the top and bottom side surfaces and the top and bottom surfaces of the X-shaped structure 115 are flat, as shown in FIG. 5. In this embodiment, the side cross-section has a double wedge shape converging both toward the back and toward the front side, as shown in FIG. 6C. The wedge height 169d is between 1 and 2 millimeters. Referring to FIG. 7, in other embodiments, interbody cage 160 includes a threaded aperture 161 positioned in the corner or the side surface of the cage 160 for engaging a tool for positioning the interbody cage 110 at a 45 degree angle or sidewise between the vertebras 82, 84. Referring to FIG. 8A, the ALIF assembly 100 is inserted anteriorly along the direction of arrow 92. In other embodiments, the assembly is a posterior lumbar interbody fusion (PLIF) assembly 200 and is inserted posteriorly along the direction of arrow 94. Referring to FIG. 9A and FIG. 9B, in this embodiment, the interbody cage 200 is bullet shaped and includes narrow top and bottom surfaces 202a, 202b, a convex shaped front surface 204a and a flat or concave shaped back surface 204b and wide side surfaces 206a, 206b. The interior is again hollow and includes an X-shaped structure 205 forming opening 203a, 203b, 203c, 203d with the top, front, bottom and back surfaces. The top and bottom surfaces have surface teeth 245 used to engage the adjacent vertebral surfaces. In other embodiments surfaces 202a, 2022b include ridges, serrations, grooves, or spikes. The back surface 204b includes a threaded aperture 201 used for engaging a tool for positioning the interbody cage 200 between the vertebras 82, 84. Within apertures 203a-203d, material that promotes bone growth is deposited. In one example the interbody cage 200 is made of Polyetheretherketone (PEEK) and has a length 209a about 25 millimeters, width 209b about 8 millimeters and height 209c about 13 millimeters.

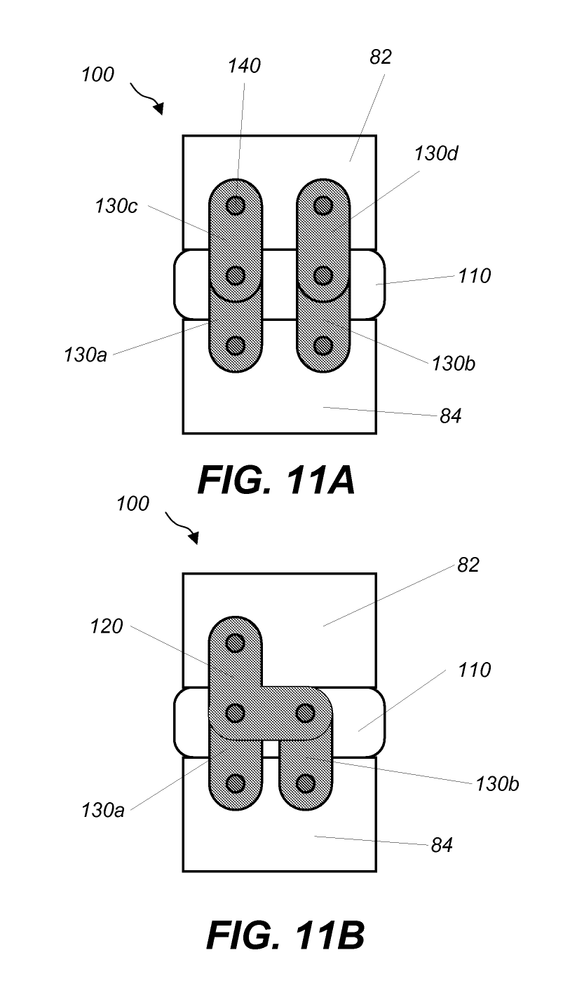

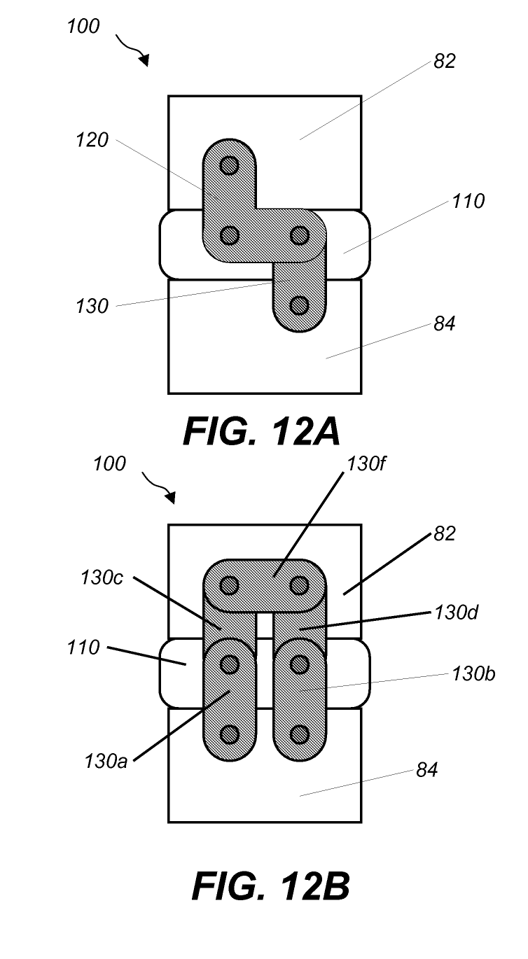

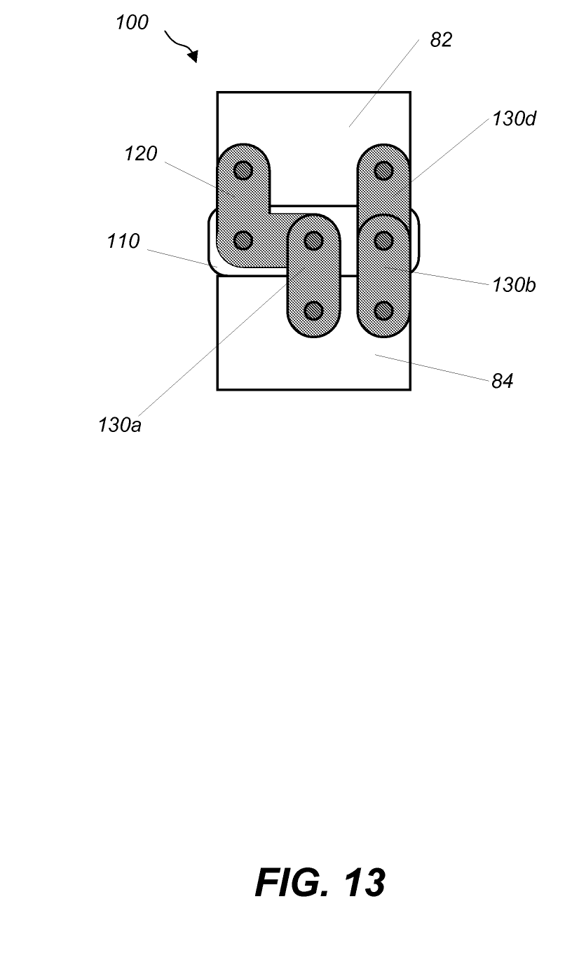

Other embodiments are within the scope of the following claims. For example, plates 120 and 30 may have an adjustable length or may be attached to the vertebras and/or interbody cage with pins or hooks. More than two plates may be used to attach the interbody cage to the vertebras. Referring to FIG. 11A-FIG. 13, various combinations of plate configurations and number of plates may be used. In particular, referring to FIG. 11A, plate 130a has a first end 136b attached to a location in vertebra 84 and a second end 130a attached to the interbody cage 110 and to plate 130c. Plate 130c has a first end 137a attached to a location in vertebra 82 and a second end 137b attached to the interbody cage 110 and to end 136a of plate 130a. Referring to FIG. 11B, L-shaped plate 120 has a first end 126a attached to a location of vertebra 82 and two additional points 126b and 126c attached to the interbody cage 110 and to ends 136a and 137a of plates 130a and 130b, respectively. End 136b of plate 130a and end 137b of plate 130b are attached to two different locations of vertebra 84. More than two adjacent vertebras may be fused together by utilizing additional interbody cages and plates.

Several embodiments of the present invention have been described. Nevertheless, it will be understood that various modifications may be made without departing from the spirit and scope of the invention. Accordingly, other embodiments are within the scope of the following claims.

What is claimed is:

1. An interbody spinal fusion assembly for fusing a first vertebra to a second vertebra wherein said first vertebra is adjacent to said second vertebra comprising:
an interbody cage configured to be implanted between said first and second vertebras, wherein said interbody cage comprises through-apertures configured to be filled with bone fusing material;
a first plate comprising an L-shaped body having a first end configured to be attached to a first location of said interbody cage and a second end configured to be attached to a first location of said first vertebra; and
a second plate comprising a first end configured to be attached to a second location of said interbody cage and a second end configured to be attached to a first location of said second vertebra.

2. The spinal fusion assembly of claim 1, wherein said first plate further comprises a corner formed between said first and second ends and configured to be attached to a second location of said first vertebra.

3. The spinal fusion assembly of claim 1, wherein said first plate further comprises a corner formed between said first and second ends and configured to be attached to a third location of said interbody cage.

4. The spinal fusion assembly of claim 1, wherein said first location of said interbody cage coincides with said second location of said interbody cage.

5. The spinal fusion assembly of claim 1, wherein said second plate comprises an I-shaped body.

6. The spinal fusion assembly of claim 1, wherein said second plate comprises a T-shaped body.

7. The spinal fusion assembly of claim 6, wherein said second plate comprises a third end configured to be attached to a second location of said second vertebra.

8. The spinal fusion assembly of claim 1, wherein said second plate comprises one of H-shaped body, V-shaped body, L-shaped body or X-shaped body.

9. The spinal fusion assembly of claim 1, further comprising a third plate comprising a first end configured to be attached to a fourth location of said interbody cage and a second end configured to be attached to a third location of said second vertebra.

10. The spinal fusion assembly of claim 1, wherein said interbody cage comprises a hollow body having a front insertion wall, a back trailing wall, left and right side walls and an X-shaped structure placed in said hollow body and having ends connected to locations in said side walls and said front and back walls.

11. The spinal fusion assembly of claim 10, wherein said interbody cage comprises an aperture configured to engage a tool used to insert said interbody cage between said first and second vertebras and wherein said aperture is formed in one of said walls or at a corner between two of said walls.

12. The spinal fusion assembly of claim 10, wherein said through-apertures of said interbody cage are formed between said X-shaped structure and said walls.

13. The spinal fusion assembly of claim 1, wherein said interbody cage comprises top and bottom surfaces having protrusions configured to engage surfaces of said first and second vertebras and wherein said protrusions comprise one of ridges, grooves, teeth, serrations, or spikes.

14. The spinal fusion assembly of claim 1, wherein said interbody cage comprises one of metal, ceramic, bone, PEEK, plastic, stainless steel, titanium, gold, silver, nickel, alloys thereof, polymer, composites, absorbable material, metal matrix material, polycarbon coating or combinations thereof.

15. The spinal fusion assembly of claim 1, wherein said bone fusing material comprises one of hydroxyapatite, hydroxyapatite tricalcium, fibronectin, morphogenic proteins, bone growth promoting material, or combinations thereof.

16. The spinal fusion assembly of claim 1, further comprising one or more screws for attaching said plates to said interbody cage and said vertebras.

17. The spinal fusion assembly of claim 1 wherein said interbody cage comprises a bullet shaped front insertion wall.

18. The spinal fusion assembly of claim 1, wherein said interbody cage comprises a width smaller than its height.

19. The spinal fusion assembly of claim ,1 wherein any of said plates comprises an adjustable length.

20. A method for fusing a first vertebra to a second vertebra wherein said first vertebra is adjacent to said second vertebra comprising:
inserting an interbody cage between said first and second vertebras, wherein said interbody cage comprises through-apertures configured to be filled with bone fusing material;
providing a first plate comprising an L-shaped body having first and second ends and a corner formed between said first and second ends and attaching said first end to a first location of said interbody cage and said second end to a first location of said first vertebra; and
providing a second plate comprising first and second ends and attaching said first end to a second location of said interbody cage and said second end to a first location of said second vertebra.

21. The method of claim 20, further comprising attaching said corner to a second location of said first vertebra.

22. The method of claim 20, further comprising attaching said corner to a third location of said interbody cage.

23. The method of claim 20, wherein said first location of said interbody cage coincides with said second location of said interbody cage.

24. The method of claim 20, wherein said second plate comprises an I-shaped body.

25. The method of claim 20, wherein said second plate comprises a T-shaped body.

26. The method of claim 25, wherein said second plate comprises a third end configured to be attached to a second location of said second vertebra.

27. The method of claim 26, wherein said second plate comprises one of H-shaped body, V-shaped body, L-shaped body or X-shaped body.

28. The method of claim 20, further comprising providing a third plate comprising first and second ends and attaching said first end to a fourth location of said interbody cage and said second end to a third location of said second vertebra.

29. The method of claim 20, wherein said interbody cage comprises a hollow body having a front insertion wall, a back trailing wall, left and right side walls and an X-shaped structure placed in said hollow body and having ends connected to locations in said side walls and said front and back walls.

30. The method of claim 29, wherein said interbody cage comprises an aperture configured to engage a tool used to insert said interbody cage between said first and second vertebras and wherein said aperture is formed in one of said walls or at a corner between two of said walls.

31. The method of claim 29, wherein said through-apertures of said interbody cage are formed between said X-shaped structure and said walls.

32. The method of claim 20, wherein said interbody cage comprises top and bottom surfaces having protrusions configured to engage surfaces of said first and second vertebras and wherein said protrusions comprise one of ridges, grooves, teeth, serrations, or spikes.

33. The method of claim 20, wherein said interbody cage comprises one of metal, ceramic, bone, PEEK, plastic, stainless steel, titanium, gold, silver, nickel, alloys thereof, polymer, composites, absorbable material, metal matrix material, polycarbon coating or combinations thereof.

34. The method of claim 20, wherein said bone fusing material comprises one of hydroxyapatite, hydroxyapatite tricalcium, fibronectin, morphogenic proteins, bone growth promoting material, or combinations thereof.

35. The method of claim 20, further comprising providing one or more screws for attaching said plates to said interbody cage and said vertebras.

36. The method of claim 20, wherein said interbody cage comprises a bullet shaped front insertion wall.

37. The method of claim 20, wherein said interbody cage comprises a width smaller than its height.

38. The method of claim 20, wherein any of said plates comprises an adjustable length and said method comprises adjusting said plate length.

39. An interbody spinal fusion assembly for fusing a first vertebra to a second vertebra wherein said first vertebra is adjacent to said second vertebra comprising:
an interbody cage configured to be implanted between said first and second vertebras, wherein said interbody cage comprises through-apertures configured to be filled with bone fusing material;
a first plate comprising a body having a first end configured to be attached to a first location of said interbody cage and a second end configured to be attached to a first location of said first vertebra;
a second plate comprising a first end configured to be attached to a second location of said interbody cage and a second end configured to be attached to a first location of said second vertebra; and
wherein said first location of said interbody cage coincides with said second location of said interbody cage.
